(19) United States
(12) Patent Application Publication
(10) Pub. No.: US 2025/0381562 A1
(43) Pub. Date: Dec. 18, 2025

(54) SYSTEM AND METHOD FOR EXTRACTION OF REPRESENTATIVE FLUID MIXTURES FROM AN INTERMEDIATE MIXING VESSEL

(71) Applicant: CHEVRON U.S.A. INC., San Ramon, CA (US)

(72) Inventors: Richard Allen Guilbeau, Katy, TX (US); James Malcolm Strawn, JR., Houston, TX (US); Gary M. Potten, Houston, TX (US)

(21) Appl. No.: 19/237,896

(22) Filed: Jun. 13, 2025

Related U.S. Application Data
(60) Provisional application No. 63/660,269, filed on Jun. 14, 2024.

(51) Int. Cl. B01L 3/00; B01F 23/40; B01F 23/43; B01F 35/43; B01F 35/75; B01F 101/23

(52) U.S. Cl. CPC B01L 3/502; B01F 23/405; B01F 23/43; B01F 35/43; B01F 35/7547; B01F 2101/23; B01F 2215/044; B01L 2200/026; B01L 2300/0809; B01L 2300/0858; B01L 2400/06

(57) ABSTRACT

A laboratory testing system includes a shear mixer and an intermediate container sized to surround a mixing component of the shear mixer therein. The intermediate container includes an opening at an upper end, a lower surface, one or more side walls extending upward from the lower surface and a port extending through a wall of the one or more side walls or through the lower surface. The port provides access to the intermediate container for removal of a portion of a fluid mixture from the intermediate container during mixing of the fluid in the intermediate container by the shear mixer.

### CROSS REFERENCE TO RELATED APPLICATION

This application claims the benefit of U.S. Provisional Patent Application No. 63/660,269, filed on Jun. 14, 2024, entitled “System and Method for Extraction of Representative Fluid Mixtures from an Intermediate Mixing Vessel,” which is hereby incorporated by reference in its entirety for all purposes.

### FIELD OF THE INVENTION

The present disclosure is directed generally to the extraction of representative samples of fluid mixtures and, more particularly, to a laboratory apparatus and procedure for the extraction of representative samples of fluid mixtures from an intermediate mixing vessel for laboratory analysis.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Crude oil sampling from oil and gas operations is used to determine various metrics such as water/solids concentration within the crude oil, and mismeasuring of water and sediment in the crude oil can negatively impact revenue accounting. As such, it is important to obtain accurate representative samples of the crude oil for analysis. Sometimes, large representative samples are taken from a sampling point in the field and transported to a laboratory in an intermediate container. In the laboratory setting, however, obtaining representative aliquots of oil and water mixtures for analysis from the intermediate container can be difficult due to the speed of mixture separation of the water and oil in the mixture. Mixing and subsequent pouring or pipetting the mixture from the intermediate container into smaller sampling containers (e.g., centrifuge tubes), according to existing laboratory procedures, can result in aliquots that are not repeatably representative of the mixture in the intermediate container. While this effect is relatively minor for crude oil with low water concentrations (e.g., ˜2% by volume), the results are worse for representative aliquots of crude oil with high concentrations of water (e.g., >15% by volume). It is now recognized that a need exists for a laboratory apparatus and procedure for obtaining representative aliquots of crude oil from an intermediate container with high accuracy and repeatability.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In accordance with aspects of the disclosure, a laboratory testing system includes a shear mixer and an intermediate container sized to surround a mixing component of the shear mixer therein. The intermediate container includes an opening at an upper end, a lower surface, one or more side walls extending upward from the lower surface and a port extending through a wall of the one or more side walls or through the lower surface. The port provides access to the intermediate container for removal of a portion of a fluid mixture from the intermediate container during mixing of the fluid in the intermediate container by the shear mixer.

In accordance with aspects of the disclosure, a laboratory testing method includes: receiving a liquid/liquid mixture in an intermediate container having a lower surface and one or more side walls extending upward from the lower surface; mixing the liquid/liquid mixture with a shear mixer extending into the intermediate container through an opening at an upper end of the intermediate container to evenly mix the liquids of the mixture in the intermediate container; and via a port extending through a side wall of the one or more side walls or through the lower surface of the intermediate container, enabling removal of a portion of the liquid/liquid mixture from the intermediate container while continuously mixing the liquid/liquid mixture.

In accordance with aspects of the disclosure, a laboratory testing method includes: receiving a water/oil mixture in an intermediate container having a lower surface and one or more side walls extending upward from the lower surface; mixing the water/oil mixture with a shear mixer extending into the intermediate container through an opening at an upper end of the intermediate container to evenly mix the water and oil of the mixture in the intermediate container; opening a valve coupled to a port extending through a side wall of the one or more side walls or through the lower surface to remove a portion of the water/oil mixture from the intermediate container while continuously mixing the water/oil mixture; and receiving the portion of the water/oil mixture directly from the valve into a test container.

In accordance with aspects of the disclosure, a laboratory testing method includes: receiving a water/oil mixture in an intermediate container having a lower surface and one or more side walls extending upward from the lower surface; mixing the water/oil mixture with a shear mixer extending into the intermediate container through an opening at an upper end of the intermediate container to evenly mix the water and oil of the mixture in the intermediate container; and using a syringe positioned through a septum in a side wall of the one or more side walls of the intermediate container, withdrawing a portion of the water/oil mixture from the intermediate container while continuously mixing the water/oil mixture.

### BRIEF DESCRIPTION OF DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, reference numerals designate like or corresponding, but not necessarily identical, elements.

FIG. 1 is a schematic illustration of a laboratory setup for providing a representative sample of a fluid mixture from an intermediate container into a sample container, in accordance with an embodiment of the present disclosure.

FIG. 2 is a photograph illustrating an intermediate container that may be used in the laboratory setup of FIG. 1, in accordance with an embodiment of the present disclosure.

FIG. 3 is a schematic illustration of another laboratory setup for providing a representative sample of a fluid mixture from an intermediate container into a sample container, in accordance with an embodiment of the present disclosure.

FIG. 4 is a schematic illustration of another laboratory setup for providing a representative sample of a fluid mixture from an intermediate container into a sample container, in accordance with an embodiment of the present disclosure.

FIG. 5 is a process flow diagram illustrating a laboratory testing method for obtaining representative samples of a fluid mixture from an intermediate container, in accordance with an embodiment of the present disclosure.

FIG. 6 is a process flow diagram illustrating a laboratory testing method for obtaining representative samples of a fluid mixture for centrifuge analysis, in accordance with an embodiment of the present disclosure.

FIG. 7 is a process flow diagram illustrating a laboratory testing method for obtaining representative samples of a fluid mixture for Karl Fischer analysis, in accordance with an embodiment of the present disclosure.

### DETAILED DESCRIPTION OF THE INVENTION

The present disclosure is directed to laboratory equipment, apparatus, and procedures for measuring water concentrations in fluid samples (e.g., crude oil samples). The disclosed equipment, apparatus, and procedures facilitate mixing of water and oil samples and extraction of representative aliquots while the mixing system is engaged, resulting in accurate and repeatable representative crude oil samples. The disclosed laboratory equipment includes an intermediate container that may be used for mixing samples in conjunction with a stand shear mixer. The term “intermediate container” refers to any container that may be used to receive a crude oil sample from a sampling point (e.g., sample vessel) at a field location where oil and gas operations take place, for transportation to a laboratory for further testing of the crude oil sample.

The intermediate container may include a sample valve, a septum, or both in the body allowing for extraction of representative aliquots directly into a smaller sampling container (e.g., centrifuge tube, syringe, or other container) while the mixer is running. The intermediate container is configured to facilitate collection of a portion of the water/oil mixture having a substantially equivalent volumetric percentage of water as the fluid mixture being mixed in the intermediate container. In an example, the fluid mixture has a volumetric percentage of water of at least 15%. In another example, the fluid mixture has a volumetric percentage of water less than approximately 15%. The disclosed laboratory equipment, apparatus, and procedures may be particularly useful in determining water content in light crude oil that separates easily and/or in crude oil with a high watercut ratio.

The disclosed laboratory equipment, apparatus, and procedures may be used to analyze test samples of crude oil in a laboratory setting for various purposes. For example, the laboratory equipment, apparatus, and procedures may be used for purposes of water content determination to determine revenue. Additionally, or alternatively, the laboratory equipment, apparatus, and procedures may be used for purposes of validating performance of instruments in the field (e.g., online watercut analyzers (WCAs)).

The term “fluid mixture” is used throughout the following description. The fluid mixture may be any mixture of at least two liquids (e.g., a “liquid/liquid mixture”). The at least two liquids may be naturally separable from each other, making it difficult to obtain representative aliquots using existing equipment. In an example, the fluid mixture as described below may be a fluid mixture in which the two mixed liquids are water and oil (e.g., a “water/oil mixture”). It should be understood that the disclosed laboratory equipment, while described below as being used to obtain representative aliquots of a water/oil mixture, may similarly be used for obtaining representative aliquots of any fluid mixture (e.g., any separable fluid mixture) and is not necessarily limited to use with a water/oil mixture.

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of ±10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

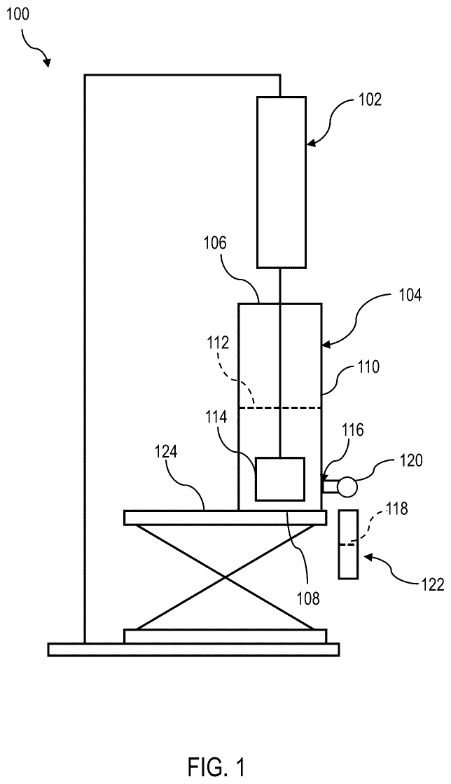

Turning now to the drawings, FIG. 1 illustrates an example laboratory testing system 100, in accordance with an embodiment of the present disclosure. The system 100 may be used to obtain representative samples of a fluid mixture (e.g., a mixture of oil and water (e.g., crude oil)) from a larger container (e.g., intermediate container) for laboratory testing. The system 100 includes a shear mixer 102 and an intermediate container 104. The intermediate container 104 has an opening 106 at its upper end, a lower surface 108 at its lower end, and one or more side walls 110 extending upward from the lower surface 108 toward the opening 106. The intermediate container 104 may be any desired size or shape and have any desired volume (e.g., cylindrical, rectangular prism, spherical, etc.). The intermediate container 104 is designed to hold a fluid mixture, e.g., a mixture of at least water and oil, referred to herein as a “water/oil mixture” 112. It should be noted that the water/oil mixture 112 may include other components such as, for example, sediment or contaminants in addition to water and oil. The intermediate container 104 is sized to surround a mixing component 114 (e.g., one or more paddles, blades, etc.) of the shear mixer 102. For example, the intermediate container 104 may receive the mixing component 114 into the intermediate container 104 through the opening 106, with the intermediate container 104 and the shear mixer 102 being separate components. As another example, the shear mixer 102 may be integral with the intermediate container 104 with the mixing component 114 located inside the volume of the intermediate container 104.

The intermediate container 104 also includes a port 116 extending through a side wall 110, as shown. The port 116 provides access to the intermediate container 104 for removal of a portion 118 of the water/oil mixture 112 from the intermediate container 104 during mixing of the water/oil mixture 112 in the intermediate container 104 by the shear mixer 102. As illustrated, the system 100 may further include a valve 120 fluidly coupled to the port 116 of the intermediate container 104. The valve 120 is located along the side wall 110 of the intermediate container 104, as shown. The valve 120 may be a manually operated valve, such as a spigot, or an electric or hydraulic operated valve whose actuation is controlled by a control system. The valve 120 may be incorporated into the port 116.

As shown, the sampled portion 118 of the water/oil mixture may be output through the port 116/valve 120 into a test container 122 used for further testing of the portion 118. The test container 122 may include a centrifuge tube in certain embodiments. The centrifuge tube may be a glass, eight inch, 100 mL centrifuge tube with 1 mL graduations marked on its side. Other types, materials, sizes, and volumes of centrifuge tubes may be used as well. The port 116/valve 120 are used to draw off fluid directly into centrifuge tube(s) from the intermediate container during mixing. It may be desirable to fill each centrifuge tube to approximately the same level with the water/oil mixture from the intermediate container 104. The filling may be performed continually in one opening/closing cycle of the valve 120 to fill the graduated centrifuge tube to a desired point. In other instances, the filling may be performed in multiple filling cycles (e.g., in multiple opening/closing cycles of the valve 120), with breaks in between to let the water/oil mixture settle in the centrifuge tube.

In addition, the system 100 may include a stand 124 upon which the intermediate container 104 is positioned to elevate the intermediate container 104 during mixing of the water/oil mixture and filling of the test container 122. The stand 124 provides space beneath the port 116/valve 120 for the test container 122 to be placed for direct transfer of the portion 118 of the water/oil mixture from the intermediate container 104 to the test container 122. In some embodiments, the stand may be adjustable, although this is not required. Any structural component capable of supporting and elevating the intermediate container 104 with the water/oil mixture 112 may be used for the stand 124.

Since the shear mixer 102 is mixing the water/oil mixture 112 in the intermediate container 104 throughout the sampling process, the system 100 may provide the portion 118 of the water/oil mixture from the intermediate container 104 to the test container 122 such that the output portion 118 has a volumetric percentage of water that is approximately equivalent to that of the initial water/oil mixture 112 in the intermediate container 104. In some embodiments, the water/oil mixture 112, and resulting portion 118 of the water/oil mixture removed from the intermediate container 104, may have a volumetric percentage of water between approximately 15% and approximately 95%. At such large water concentrations, the disclosed intermediate container 104 and test system 100 provide an accurate and repeatable representative sampling of the water/oil mixture 112 (i.e., consistent volumetric percentage of water between the sampled portion 118 and the water/oil mixture in the intermediate container 104).

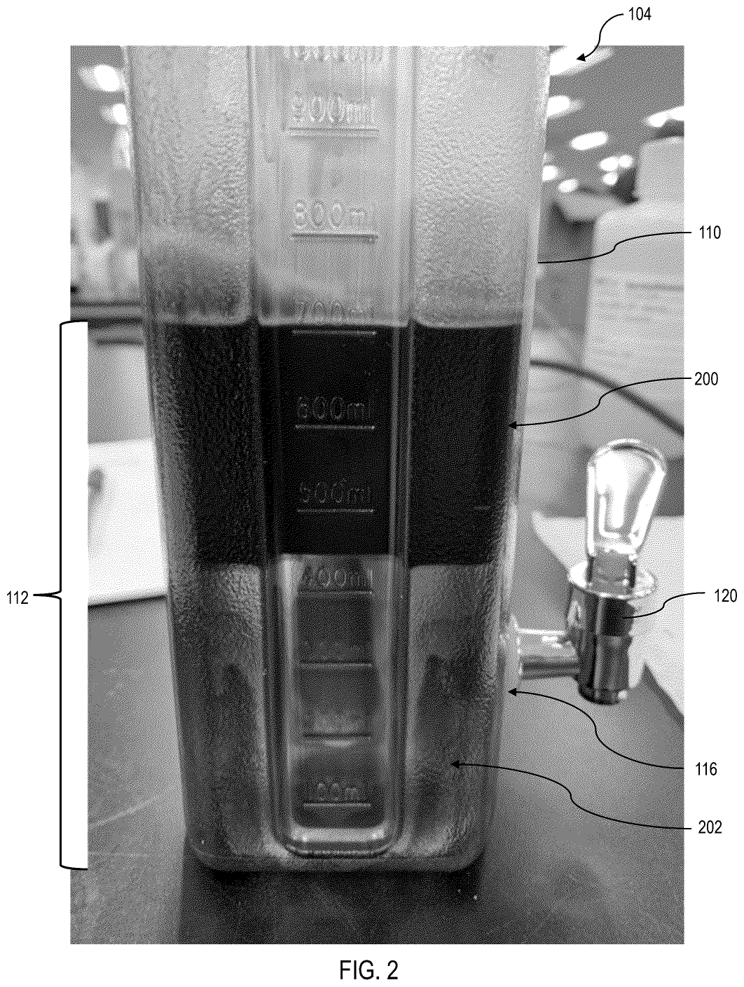

FIG. 2 is a photograph showing an example intermediate container 104 that may be used in the system of FIG. 1. As illustrated, the intermediate container 104 may be constructed from plastic, and the valve 120 may be a small sample take-off spigot. Other types of valves may be used in other embodiments. The port 116 and valve 120 may be placed at any desired location on the side wall 110 of the intermediate container 104. It may be desirable to locate the port 116 and valve 120 along the lower two-thirds, or more particularly the lower half, or more particularly the lower fourth, of the height of the intermediate container 104, so that less water/oil mixture 112 is needed in the intermediate container 104 to allow the filling of test containers from the port 116/valve 120. As shown, the intermediate container 104 may have a total volume of 1 Liter and may be graduated with markings along one or more of its side walls. The intermediate container 104 may be constructed from clear plastic or glass, thus providing a visual of the water/oil mixture 112 therein. In FIG. 2, the water/oil mixture 112 is separated with the oil 200 above the water 202 since a mixer is not currently mixing the water and oil. During filling of test containers through the port 116/valve 120, however, the water/oil mixture 112 will be mixed so that it is substantially homogeneous within the container 104.

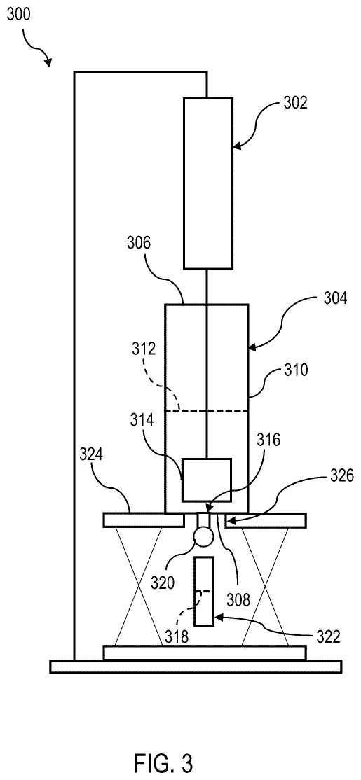

Although FIGS. 1 and 2 show an intermediate container 104 in which the port 116 and valve 120 are located along a side wall 110, other embodiments may utilize an intermediate container with a port located on an underside of the container, as shown in FIG. 3.

FIG. 3 illustrates another example laboratory testing system 300, in accordance with an embodiment of the present disclosure. The system 300 may be used to obtain representative samples of a fluid mixture (e.g., a mixture of oil and water (e.g., crude oil)) from a larger container (e.g., intermediate container) for laboratory testing. The system 300 includes a shear mixer 302 and an intermediate container 304. The intermediate container 304 has an opening 306 at its upper end, a lower surface 308 at its lower end, and one or more side walls 310 extending upward from the lower surface 308 toward the opening 306. The intermediate container 304 may be any desired size or shape and have any desired volume. The intermediate container 304 is designed to hold a fluid mixture, e.g., a water/oil mixture 312 similar to the water/oil mixture 112 described above with reference to FIG. 1. The intermediate container 304 is sized to surround a mixing component 314 (e.g., one or more paddles, blades, etc.) of the shear mixer 302, as described above similarly with reference to the intermediate container 104 of FIG. 1.

The intermediate container 304 also includes a port 316 extending through the lower surface 308, as shown. The port 316 provides access to the intermediate container 304 for removal of a portion 318 of the water/oil mixture 312 from the intermediate container 304 during mixing of the water/oil mixture 312 in the intermediate container 304 by the shear mixer 302. As illustrated, the system 300 may further include a valve 320 fluidly coupled to the port 316 of the intermediate container 304. The valve 320 is located along the lower surface 308 of the intermediate container 304. The valve 320 may be a manually operated valve, such as a spigot, or an electric or hydraulic operated valve whose actuation is controlled by a control system. The valve 320 may be incorporated into the port 316.

As shown, the sampled portion 318 of the water/oil mixture may be output through the port 316/valve 320 into a test container 322 used for further testing of the portion 318. The test container 322 may be similar to the test container 122 described above with reference to FIG. 1. The port 316/valve 320 are used to draw off fluid directly into test container(s) 322 from the intermediate container during mixing. The filling may be performed continually in one opening/closing cycle of the valve 320, or in multiple filling cycles with breaks in between to let the water/oil mixture settle in the test container 322.

The system 300 may include a stand 324 upon which the intermediate container 304 is positioned to elevate the intermediate container 304 during mixing of the water/oil mixture and filling of the test container 322. The stand 324 may be similar to the stand 124 described above with reference to FIG. 1, except the stand 324 may include an opening 326 through which the port 316/valve 320 can extend downward from the lower surface 308 of the intermediate container 304. In addition, the stand 324 may be constructed to provide space beneath the intermediate container 304 for the test container 322 to be positioned so the portion of water/oil mixture can flow directly from the intermediate container 304 into the test container 322 through the port 316/valve 320.

Since the shear mixer 302 is mixing the water/oil mixture 312 in the intermediate container 304 throughout the sampling process, the system 300 may provide the portion 318 of the water/oil mixture from the intermediate container 304 to the test container 322 such that the output portion 318 has a volumetric percentage of water that is approximately equivalent to that of the initial water/oil mixture 312 in the intermediate container 304. In some embodiments, the water/oil mixture 312, and resulting portion 318 of the water/oil mixture removed from the intermediate container 304, may have a volumetric percentage of water between approximately 15% and approximately 95%. At such large water concentrations, the disclosed intermediate container 304 and test system 300 provide an accurate and repeatable representative sampling of the water/oil mixture 312 (i.e., consistent volumetric percentage of water between the sampled portion 318 and the water/oil mixture in the intermediate container 304).

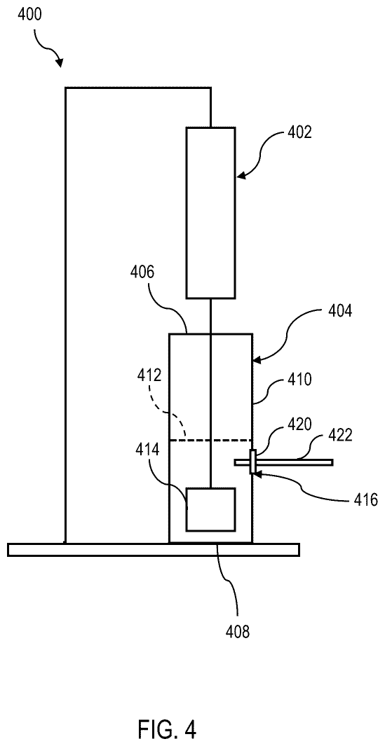

While FIGS. 1-3 show intermediate containers 104/304 in which a valve 120/320 is present for outputting representative samples, other embodiments may utilize a septum located along a side wall of the container, as shown in FIG. 4.

FIG. 4 illustrates another example laboratory testing system 400, in accordance with an embodiment of the present disclosure. The system 400 may be used to obtain relatively smaller representative samples of a fluid mixture (e.g., a mixture of oil and water (e.g., crude oil)) from a larger container (e.g., intermediate container) for laboratory testing. The system 400 includes a shear mixer 402 and an intermediate container 404. The intermediate container 404 has an opening 406 at its upper end, a lower surface 408 at its lower end, and one or more side walls 410 extending upward from the lower surface 408 toward the opening 406. The intermediate container 404 may be any desired size or shape and have any desired volume. The intermediate container 404 is designed to hold a fluid mixture, e.g., a water/oil mixture 412 similar to the water/oil mixture 112 described above with reference to FIG. 1. The intermediate container 404 is sized to surround a mixing component 414 (e.g., one or more paddles, blades, etc.) of the shear mixer 402 into the intermediate container 404 through the opening 406, as described above similarly with reference to the intermediate container 104 of FIG. 1.

The intermediate container 404 also includes a port 416 extending through a side wall 410 of the one or more side walls, as shown. The port 416 provides access to the intermediate container 404 for removal of a portion of the water/oil mixture 412 from the intermediate container 404 during mixing of the water/oil mixture 412 in the intermediate container 404 by the shear mixer 402. As illustrated, the system 400 may further include a septum 420 positioned within and/or along the port 416 of the intermediate container 404. The septum 420 may be located along the side wall 410 of the intermediate container 404, as shown. In other embodiments, the port 416/septum 420 may be positioned through the bottom surface of the intermediate container 404 (not shown). The septum 420 may be disposed within and/or along the port 416 to block the water/oil mixture from exiting the port 416.

As shown, a syringe 422 may be placed through (e.g., temporarily puncturing) the septum 420 to access and withdraw a portion of the water/oil mixture 412 from the intermediate container 404. After the representative sample has been withdrawn, the syringe 422 is removed from the septum 420, and the septum 420 again blocks the remaining part of the water/oil mixture 412 from exiting the port 416. The septum 420 may enable a syringe 422 to be placed into the intermediate container 404 to withdraw a portion of the water/oil mixture 412 multiple times without having to replace the septum 420. The septum 420 may be incorporated into the port 416. The septum 420 may be positioned proximate an end portion of the port 416 along the side wall 410 of the intermediate container 404 and then secured via a cap (not shown). The septum 420 may be a foam plug. Other types of construction of the septum 420 may be used in other embodiments. In some embodiments, the intermediate container may be a laboratory flask with a septum port.

The port 416/septum 420 are used to draw off fluid directly into one or more syringes 422 from the intermediate container 404 during mixing. The syringe 422 may be substantially smaller than and used to draw out a much smaller volume of water/oil mixture from the intermediate container 404 than the centrifuge tubes discussed above. The test syringe 422 may deposit the portion of the water/oil mixture into a testing apparatus, such as a coulometric Karl Fischer titrator for further analysis.

Since the shear mixer 402 is mixing the water/oil mixture 412 in the intermediate container 404 throughout the sampling process, the system 400 may enable a user to retrieve a small portion of the water/oil mixture from the intermediate container 404 into a syringe 422 or other test container such that the output portion has a volumetric percentage of water that is approximately equivalent to that of the initial water/oil mixture 412 in the intermediate container 404. In some embodiments, the water/oil mixture 412, and resulting portion of the water/oil mixture removed from the intermediate container 404, may have a volumetric percentage of water less than approximately 15%. At these small water concentrations, the disclosed intermediate container 404 and test system 400 may provide an accurate and repeatable representative sampling of the water/oil mixture 412 (i.e., consistent volumetric percentage of water between the sampled portion and the water/oil mixture in the intermediate container 404).

The septum 420 may be constructed from any material compatible with the fluid and syringe used that selectively allows penetration with a syringe or similar test container and subsequently closes to prevent fluid output from the septum 420. The septum 420 may be placed at any desired location on the side wall 410 of the intermediate container 404. It may be desirable to locate the septum 420 along the lower two-thirds, or more particularly the lower half, or more particularly the lower fourth, of the height of the intermediate container 404, so that less water/oil mixture 412 is needed in the intermediate container 404 to allow for drawing out samples of water/oil mixture through the septum 420. The intermediate container 404 may be constructed from clear plastic or glass, thus providing a visual of the water/oil mixture 412 therein. During withdrawal of representative samples through the septum 420 via a syringe 422, the water/oil mixture 412 will be mixed so that it is substantially homogeneous within the container 404.

For each of the intermediate containers 104, 304, and 404 described above, the intermediate container 104/304/404 may include any one or a combination of the following features. The intermediate container 104/304/404 may be sized to fit whatever size of shear mixer is used with the intermediate container 104/304/404. The intermediate container 104/304/404 may be constructed from plastic, glass, ceramic, metal, or any other fluid compatible material. The intermediate container 104/304/404 may have a volume of less than five liters. In some embodiments, the intermediate container 104/304/404 may have a volume of less than two liters. In some embodiments, the intermediate container 104/304/404 may have a rounded cross section (e.g., with one continuously rounded side wall 410). The rounded cross section may be a circular, oval, elliptical, or other cross section. In other embodiments, such as shown in FIG. 2, the intermediate container 104/304/404 may have a cross section that is square, rectangular, substantially square with rounded corners and/or curved faces, or substantially rectangular with rounded corners and/or curved faces (e.g., with four side walls 110). In some embodiments, the intermediate container 104/304/404 may be spherical in shape (e.g., with a circular cross section that varies in diameter from bottom to top of the continuous side wall). Other shapes, sizes, and materials of the intermediate container 104/304/404 may be used without departing from the scope of the present disclosure.

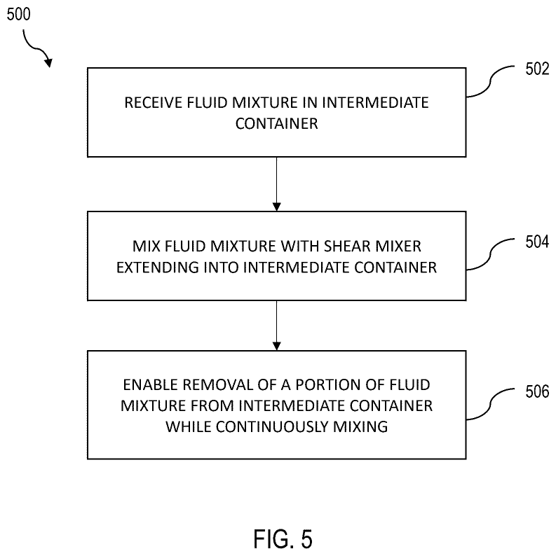

Having discussed various types of testing apparatus that may be used to provide accurate representative samples of water/oil mixtures, certain laboratory testing methods will now be described with reference to FIGS. 5-7.

FIG. 5 is a process flow diagram illustrating an example laboratory testing method 500 in accordance with an embodiment of the present disclosure. At block 502, the method 500 includes receiving a fluid mixture, i.e., liquid/liquid mixture (e.g., a water/oil mixture) in an intermediate container. As discussed above, the intermediate container has a lower surface and one or more side walls extending upward from the lower surface. The liquids (e.g., water and oil) of the mixture may be fully mixed or at least partially separated at the time it is received in the intermediate container. The fluid mixture may separate further within the intermediate container prior to mixing. At block 504, the method 500 includes mixing the fluid mixture with a shear mixer extending into the intermediate container to evenly mix the liquids (e.g., water and oil) of the mixture in the intermediate container. As discussed above, the shear mixer is extending into the intermediate container through the opening at the upper end of the intermediate container. This mixing time and shear mixer speed may be optimized for the combination of fluid being mixed and the shear mixer equipment. At block 506, the method 500 includes enabling removal of a portion of the fluid mixture from the intermediate container while continuously mixing the fluid mixture. As discussed above, the portion of the fluid mixture is removed through a port extending through a side wall (e.g., FIGS. 1, 2, 4) of the one or more side walls or through the lower surface (e.g., FIG. 3) of the intermediate container. In some embodiments, the removal of the portion of the fluid mixture may occur through a valve fluidly coupled to the port, as in FIGS. 1-3. In other embodiments, the removal of the portion of the fluid mixture may occur through a syringe placed through a septum, as in FIG. 4. As discussed above, the portion of the fluid mixture that is removed from the intermediate container may have a volumetric percentage of water substantially equivalent to that of the fluid mixture in the intermediate container.

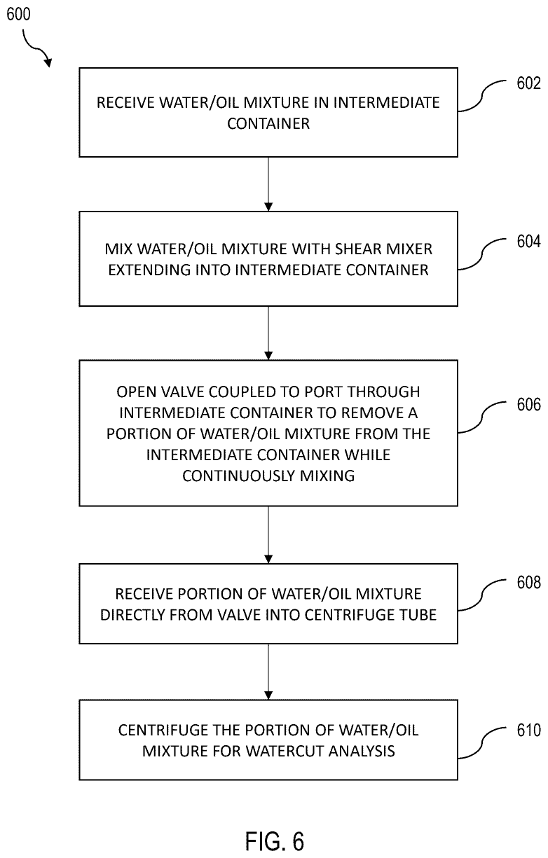

FIG. 6 is a process flow diagram illustrating an example laboratory testing method 600 in accordance with an embodiment of the present disclosure. At block 602, the method 600 includes receiving a water/oil mixture in an intermediate container. As discussed above, the intermediate container has a lower surface and one or more side walls extending upward from the lower surface. The water and oil of the mixture may be fully mixed or at least partially separated at the time it is received in the intermediate container. The water/oil mixture may separate further within the intermediate container prior to mixing. At block 604, the method 600 includes mixing the water/oil mixture with a shear mixer extending into the intermediate container to evenly mix the water and oil of the mixture in the intermediate container. As discussed above, the shear mixer is extending through the opening at the upper end of the intermediate container. At block 606, the method 600 includes opening a valve coupled to a port extending through the intermediate container to remove a portion of the water/oil mixture from the intermediate container while continuously mixing the water/oil mixture. As discussed above, the portion of the water/oil mixture is removed through the open valve coupled to the port extending through a side wall (e.g., FIGS. 1, 2) of the one or more side walls or through the lower surface (e.g., FIG. 3) of the intermediate container. At block 608, the method 600 may include receiving the portion of the water/oil mixture directly from the valve into a centrifuge tube. The centrifuge tube may be positioned directly beneath the valve and/or port through the intermediate container wall/lower surface, as shown in FIG. 1/FIG. 3. At block 610, the method 600 may include centrifuging the portion of the water/oil mixture to separate the portion of the water/oil mixture in the centrifuge tube for watercut analysis. As discussed above, the portion of the water/oil mixture that is removed from the intermediate container may have a volumetric percentage of water substantially equivalent to that of the water/oil mixture in the intermediate container. The water/oil mixture may have a volumetric percentage of water between approximately 15% and approximately 95%.

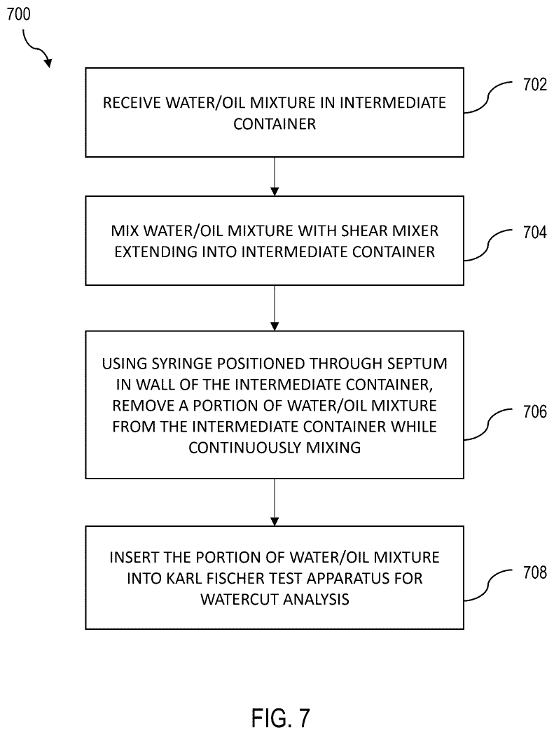

FIG. 7 is a process flow diagram illustrating an example laboratory testing method 700 in accordance with an embodiment of the present disclosure. At block 702, the method 700 includes receiving a water/oil mixture in an intermediate container. As discussed above, the intermediate container has a lower surface and one or more side walls extending upward from the lower surface. The water and oil of the mixture may be fully mixed or at least partially separated at the time it is received in the intermediate container. The water/oil mixture may separate further within the intermediate container prior to mixing. At block 704, the method 700 includes mixing the water/oil mixture with a shear mixer extending into the intermediate container to evenly mix the water and oil of the mixture in the intermediate container. As discussed above, the shear mixer is extending through the opening at the upper end of the intermediate container. At block 706, the method 700 may include using a syringe positioned through a septum (e.g., FIG. 4) in a side wall of the intermediate container to withdraw a portion of the water/oil mixture from the intermediate container while continuously mixing the water/oil mixture. At block 708, the method 700 may include inserting the portion of the water/oil mixture from the syringe into a Karl Fischer test apparatus for watercut analysis. As discussed above, the portion of the water/oil mixture that is removed from the intermediate container may have a volumetric percentage of water substantially equivalent to that of the water/oil mixture in the intermediate container. The water/oil mixture may have a volumetric percentage of water less than approximately 15%.

### Example 1

An example demonstrating the effectiveness of the disclosed test systems, apparatus, and procedures will now be provided. Example 1 demonstrates the effectiveness of the disclosed test systems, apparatus, and procedures in obtaining a representative sample of a water/oil mixture from an intermediate container that has a volumetric percentage of water substantially equivalent to that of the water/oil mixture in the intermediate container.

This example came out of a test used to assess the performance of various water cut analysis techniques for water/oil mixtures with greater than 5% water concentration. The test was performed on two different crude oils for the oil portion of the mixtures: West Texas Light (WTL) 48.6 API; and South Louisiana Intermediate (SLI) 31.5 API. The test was performed with a 1% saline water solution for the water portion of the mixtures. The water cut analysis tests were conducted using industry standard methods and equipment in water concentrations from 15% to 95%, in 10% increments. Table 1 shows the composition of the water/oil mixtures that were tested for each of the two crude oils.

TABLE 1

| Nominal | Oil cc | Water cc | Actual W % | Total Vol |
|---|---|---|---|---|
| 15% | 500 | 90 | 15.25% | 590 |
| 25% | 400 | 130 | 24.53% | 530 |
| 35% | 500 | 275 | 35.48% | 775 |
| 45% | 300 | 250 | 45.45% | 550 |
| 55% | 320 | 400 | 55.56% | 720 |
| 65% | 250 | 450 | 64.29% | 700 |
| 75% | 160 | 500 | 75.76% | 660 |
| 85% | 90 | 500 | 84.75% | 590 |
| 95% | 25 | 500 | 95.24% | 525 |

The following procedure was used for each water/oil mixture:

Oil baseline: Determine the baseline water concentration of the “dry” oil. A Karl Fischer titration method and apparatus was used to measure and record the water content from the “dry” oil. This measurement was repeated three times to ensure repeatability. The density of the oil was determined using a lab densitometer.

Select/Weigh glassware: Select the graduated cylinders required for the desired water concentration of the sample and, if not done so for a previous concentration, weigh the clean dry glassware and record.

Measure oil and water: Pour the required water and oil components in the glassware to the volumes to generate a water/oil mixture with the concentration from Table 1 and record the volume.

Weigh glassware with oil and water: Weigh the oil and water filled cylinders and record the mass.

Mix concentration: Pour the measured oil and saline water volumes into an intermediate container having a port/valve as described above with reference to FIGS. 1-3. After the liquid bulk is transferred, allow each cylinder to drain for 30 seconds to minimize fluid loss. Using a stand shear mixer, mix the oil and water for 10 minutes at 20,000 rpm. This mixing time and shear mixer speed was optimized for the combination of fluid and equipment.

Extract samples: Draw off the required quantity of sample (during the mixing process) into centrifuge tubes. Each centrifuge tube may be filled to a volume of approximately 50 mL. The mixing takes place throughout the entire process of drawing off the sample. A total of 200 mL (e.g., four representative samples) was drawn for each test.

Measure residual mixture to provide quality check: Pour remainder of water/oil mixture from the intermediate container into a laboratory graduated cylinder and record water/oil levels after 10 minutes. Then, measure the overall volume and watercut ratio of the remainder water/oil mixture.

Results were that the above process provided a direct verification of the water/oil volumes and water/oil ratios with respect to the centrifuge aliquots analysis and the total starting water/oil volumes and water/oil ratios. Upon pouring the leftover water/oil mixture into the graduated cylinder and letting it separate, it was confirmed that the volume and watercut ratio were what was expected based on the initial volume and watercut ratio for the mixture.

Additional steps were performed in the test, including centrifuging samples using different standard techniques and verifying the efficiency of the centrifuge separation by determining the residual water in the centrifuge tube oil layer with a Karl Fischer titration apparatus. However, for purposes of evaluating the disclosed systems, apparatus, and methods for providing representative samples, the quality check (provided based on the measured residual mixture in the intermediate container) indicated that the disclosed intermediate container with the port formed therein yielded an accurate and repeatable aliquot sample for further testing.

It will be understood that various modifications may be made to the embodiments disclosed herein. Therefore, the above description should not be construed as limiting, but merely as exemplifications of example embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A laboratory testing system, comprising:
a shear mixer; and
an intermediate container sized to surround a mixing component of the shear mixer therein, wherein the intermediate container comprises:
an opening at an upper end;
a lower surface;
one or more side walls extending upward from the lower surface; and
a port extending through a wall of the one or more side walls or through the lower surface, the port providing access to the intermediate container for removal of a portion of a fluid mixture from the intermediate container during mixing of the fluid in the intermediate container by the shear mixer.

2. The system of claim 1, further comprising a valve fluidly coupled to the port of the intermediate container, the valve being located along the side wall or the lower surface of the intermediate container.

3. The system of claim 1, further comprising a septum disposed within and/or along the port and located along the side wall of the intermediate container.

4. The system of claim 1, further comprising a stand upon which the intermediate container is positioned to elevate the intermediate container during mixing of the fluid mixture.

5. The system of claim 1, wherein the intermediate container has a rounded cross section.

6. The system of claim 1, wherein the intermediate container has a cross section that is rectangular, or substantially rectangular with rounded corners and/or curved faces.

7. The system of claim 1, wherein the intermediate container is configured to enable collection of the portion of the fluid mixture having a substantially equivalent volumetric percentage of water as the fluid mixture being mixed in the intermediate container.

8. The system of claim 7, wherein the fluid mixture has a volumetric percentage of water up to approximately 95%.

9. The system of claim 1, wherein the fluid mixture is a water/oil mixture.

10. A laboratory testing method, comprising:
receiving a liquid/liquid mixture in an intermediate container having a lower surface and one or more side walls extending upward from the lower surface;
mixing the liquid/liquid mixture with a shear mixer extending into the intermediate container through an opening at an upper end of the intermediate container to evenly mix the liquids of the mixture in the intermediate container; and
via a port extending through a side wall of the one or more side walls or through the lower surface of the intermediate container, enabling removal of a portion of the liquid/liquid mixture from the intermediate container while continuously mixing the liquid/liquid mixture.

11. The method of claim 10, comprising enabling the removal of the portion of the liquid/liquid mixture through a valve fluidly coupled to the port, the valve being located along the side wall or the lower surface of the intermediate container.

12. The method of claim 10, comprising enabling the removal of the portion of the liquid/liquid mixture via a syringe placed through a septum located along the side wall of the intermediate container.

13. The method of claim 10, wherein the portion of the liquid/liquid mixture has a volumetric percentage of water substantially equivalent to that of the liquid/liquid mixture in the intermediate container.

14. The method of claim 10, wherein the liquid/liquid mixture is a water/oil mixture.

15. A laboratory testing method, comprising:
receiving a water/oil mixture in an intermediate container having a lower surface and one or more side walls extending upward from the lower surface;
mixing the water/oil mixture with a shear mixer extending into the intermediate container through an opening at an upper end of the intermediate container to evenly mix the water and oil of the mixture in the intermediate container;
opening a valve coupled to a port extending through a side wall of the one or more side walls or through the lower surface to remove a portion of the water/oil mixture from the intermediate container while continuously mixing the water/oil mixture; and
receiving the portion of the water/oil mixture directly from the valve into a test container.

16. The method of claim 15, further comprising centrifuging the portion of the water/oil mixture to separate the portion of the water/oil mixture in the test container for watercut analysis.

17. The method of claim 15, wherein the water/oil mixture has a volumetric percentage of water of at least approximately 15%.

18. A laboratory testing method, comprising:
receiving a water/oil mixture in an intermediate container having a lower surface and one or more side walls extending upward from the lower surface;
mixing the water/oil mixture with a shear mixer extending into the intermediate container through an opening at an upper end of the intermediate container to evenly mix the water and oil of the mixture in the intermediate container; and
using a syringe positioned through a septum in a side wall of the one or more side walls of the intermediate container, withdrawing a portion of the water/oil mixture from the intermediate container while continuously mixing the water/oil mixture.

19. The method of claim 18, further comprising inserting the portion of the water/oil mixture into a Karl Fischer test apparatus for watercut analysis.

20. The method of claim 18, wherein the water/oil mixture has a volumetric percentage of water less than approximately 15%.
